(19) United States
(12) Patent Application Publication
(10) Pub. No.: US 2020/0107941 A1
(43) Pub. Date: Apr. 9, 2020

(54) ORIENTATION GUIDE

(71) Applicant: DEPUY IRELAND UNLIMITED COMPANY, COUNTY CORK (IE)

(72) Inventors: IAN LESLIE, LEEDS (GB); STEPHANIE PRINCE, WAKEFIELD (GB); PHILIP WRIGHT, (GB); DUNCAN YOUNG, MELBOURN (GB)

(21) Appl. No.: 16/500,845

(22) PCT Filed: Apr. 9, 2018
(86) PCT No.: PCT/EP2018/059064

§ 371 Date: Oct. 4, 2019

(30) Foreign Application Priority Data
Apr. 12, 2017 (GB) 1705918.9

(51) Int. Cl. A61F 2/46; A61F 2/36; A61F 2/34

(52) U.S. Cl. CPC A61F 2/4609; A61F 2/3609; A61F 2/4684; A61F 2002/4668; A61F 2/4657; A61F 2002/4687; A61F 2002/3625; A61F 2/34

(57) ABSTRACT

An orientation guide (400, 450, 452, 470, 472, 480, 482, 484, 486, 488, 490, 492, 494) and method for visually assessing the orientation of an acetabular cup implanted in a patient are described. The orientation guide comprises a body (402) adapted to be mounted on a femoral neck, a support (404) extending from the body and an alignment member (406, 483, 491) mounted on the support. The alignment member is tilted relative to the body and configured to extend along an alignment axis parallel to a target anteversion angle of the acetabular cup when the orientation guide in use is attached to the femoral neck and the femoral neck includes a femoral head which is received in the acetabular cup to form a hip joint.

The present invention relates to hip surgery and in particular to instruments and methods which may be used during hip surgery to visually assess acetabular cup orientation.

A variety of methods of hip surgery are generally known. The hip joint may generally be considered a ball and socket joint in which the head of the femur articulates within the acetabular cavity of the pelvis. Some methods of hip surgery may involve the replacement of one or more parts of the hip joint with one or more prosthetic components. This may be to replace damaged, worn, diseased or otherwise imperfect parts of the hip joint including the respective articulating surfaces of the acetabular cavity and/or femoral head.

Different surgical procedures may involve replacement of a part of the acetabulum or the femoral head or both. Some procedures, sometimes referred to as resurfacing procedures, may involve replacement of only the articulating surface of the femoral head. Other procedures may involve replacement of the entire femoral head. Such procedures often also use a femoral or stem component which is implanted in the resected femur and having a neck to which the femoral head is attached. In some procedures, a prosthetic cup may be implanted in a prepared acetabular cavity to provide a cavity in which the femoral head or prosthetic femoral head may articulate when the joint is reduced. Surgical procedures in which both the acetabulum and at least a part of the femoral head are replaced with prosthetic components are generally referred to as total hip replacement procedures.

During hip surgery procedures, some surgeons may sometimes use one or more trial components, which have the same geometry and size as the intended prosthetic components, so that the surgeon may trial the joint before final implantation of one or both of the prosthetic components. For example, the surgeon may use trial components to check that the size, position or orientation of one or more of the components is suitable. Other surgeons may opt not to use trial components or may use them occasionally based on their professional judgement.

One of the considerations in hip surgery is the angular orientation of the acetabular cavity. When the acetabulum is replaced with an acetabular cup, then it is often an aim of the surgeon to place the acetabular cup so that it is pointing generally in a preferred direction or range of directions. The orientation of an acetabular cup is often defined in terms of an angle of abduction, or inclination, and an angle of anteversion.

A variety of approaches have been used to try and assess the angular orientation of an acetabular cup, either a trial or a prosthesis, after placement in the acetabular cavity.

Anatomical approaches may be used in which the surgeon uses either their experience and/or a piece of instrumentation, in order to gauge, by inspection or instrumentation, the angular orientation of the acetabular cup relative to one or more anatomical features of the patient's pelvis. However, there is often limited access to the surgical site, particularly for minimally invasive approaches, and therefore this is often not easy nor accurate.

Other approaches may use markings or other features on the trial or prosthetic components in order to gauge the angular orientation of the acetabular cup relative to the patient's pelvis.

For example “The Ranawat Sign A Specific Maneuver to Assess Component Positioning in Total Hip Arthroplasty”, Lucas, David H., and Scott, Richard D., Journal of Orthopaedic Techniques, Vol. 2, No. 2, June 1994, describes a method of intraoperative assessment of component orientation for total hip arthroplasty. With the patient in the true lateral decubitus position, the femur is internally rotated without hip flexion until a flat underside of the prosthetic head (generally perpendicular to the femoral neck) is co-planar with a rim of the acetabular cup. The amount of internal rotation necessary to achieve this position is known as the Ranawat sign and relates to the combined anteversion of the acetabular and femoral components of the joint. For example a Ranawat sign of 45° may correspond to a cup anteversion of 30° and a femoral anteversion of 15°. However, any knee laxity or deformity can influence the interpretation of this value. Also, there may be difficulty in assessing the actual magnitude of the angle of internal rotation. Further, different surgeons may have different approaches to manipulating the patient's leg and also any one surgeon's approach may not easily be reproducible from patient-to-patient either by that same surgeon or by other surgeons.

WO 2009/108683 describes another approach in which markings are applied to a femoral head and in which the surgeon again applies an amount of internal rotation to the patient's leg, during trial reduction, so that the angle between the rim of the acetabular cup and various markings on the femoral head indicates the angular position of the acetabular cup relative to the patient's pelvis. A leg position is used in which the patient's leg is in full, relaxed extension at zero degrees abduction, zero degrees anteversion and approximately 15° internal rotation, or otherwise internally rotated by an amount equal to the amount of version of the natural or artificial femoral neck. Hence, this approach also requires the surgeon to apply a specific amount of internal rotation to the patient's leg. Again, it may be difficult to apply the correct amount of internal rotation, there may be inaccuracies introduced by deformities of the patient's leg and the surgical technique may be difficult to reproduce and/or reliably learn. This is particularly the case for a relatively small angle, 15°, as even a relatively small error in the amount of internal rotation, for example 5°, is a large proportion (33%) of the target internal rotation.

Other approaches and associated instrumentation are described in US 2005/0107799. An accessory for implanting a hip cup, includes a manipulable cup, a manipulation head having a hemispherical portion and a circular rim around it for aligning the manipulable cup in the acetabulum. A device for immobilizing the aligned position of the manipulable cup is provided and allows a guide to be set for alignment of a bone bur and a drive-in instrument for reaming placing the acetabular cavity and placing the cup. In another approach, a manipulable cup is located in the acetabulum and its orientation can be adjusted by a handle until a lip of the manipulable cup is parallel with an equatorial line on a femoral head or a plane on the reverse of a femoral head

Hence, apparatus and/or methods making accurate intraoperative assessment of acetabular cup placement simpler, easier and/or more reliable would be beneficial.

A first aspect of the invention provides an orientation guide for visually assessing the orientation of an acetabular cup implanted in a patient, the orientation guide comprising: a body adapted to be mounted on a femoral neck; a support extending from the body; and an alignment member mounted on the support and wherein the alignment member is tilted or inclined relative to the body and configured to extend along an alignment axis parallel to a target anteversion angle of the acetabular cup when the orientation guide in use is attached to the femoral neck and the femoral neck includes a femoral head which is received in the acetabular cup to form a hip joint.

The alignment member may be tilted or inclined by an angle of between 20° and 40° relative to the body. The alignment member may be tilted or inclined by an angle of substantially 35° relative to the body.

An axis of the alignment member and a plane of the body may subtend an acute angle of between 20° and 40°, or an acute angle of 35°.

The alignment member may be rectilinear and/or may extend in a straight line along the alignment axis.

The alignment member may extend over an alignment plane which includes the alignment axis.

The alignment member may be curved.

The alignment member may be an arc of an annulus.

The body may include a first leg and a second leg and wherein the first leg and second leg define a cavity configured to receive a femoral neck in use.

The first leg and/or the second leg may be sprung to provide a clip for releasably attaching the orientation guide to the femoral neck in use.

The orientation guide may further include a tool attachment feature for releasably attaching a tool for placing the orientation guide on the femoral neck.

The tool attachment feature may be positioned in a lateral-medial direction or an anterior-posterior direction in use.

The target anteversion angle of the acetabular cup may be 20° relative to the patient's pelvis.

The hip joint may be in an anatomical position corresponding to the patient's femur being placed in 0° of flexion/extension, 0° of adduction/abduction and 0° of internal/external rotation.

The alignment member may be configured for assessing the anteversion of the acetabular cup.

The alignment member may be configured for assessing the inclination of the acetabular cup.

The alignment member may be configured for assessing the anteversion of the acetabular cup and the inclination of the acetabular cup.

A second aspect of the invention provides a kit of parts comprising: the orientation guide of the first aspect of the invention; and a femoral neck.

The femoral neck may be a prosthetic femoral neck of a prosthetic femoral stem.

The femoral neck may be a trial femoral neck.

The kit may further comprise: a femoral cutting instrument, wherein the femoral cutting instrument includes a first attachment feature and the trial femoral neck includes a second attachment feature and wherein the trial femoral neck is releasably attachable to the femoral cutting instrument using the first attachment feature and the second attachment feature.

A third aspect of the invention provides an assembly of the kit of parts of the second aspect of the invention, wherein the orientation guide is mounted on the femoral neck.

A fourth aspect of the invention provides a method of visually assessing the orientation of an acetabular cup implanted in a patient, comprising: attaching an orientation guide having an alignment member extending along an alignment axis to a femoral neck of a hip joint including the acetabular cup; and visually inspecting the angle between the alignment member and a rim of the acetabular cup or a rim of a liner within the acetabular cup to assess how close the orientation of the acetabular cup is to a target orientation of the acetabular cup.

The femoral neck may be a trial femoral neck and the angle may be visually inspected during a trialling stage of a hip procedure.

The method may further comprise: attaching the trial femoral neck to a femoral cutting instrument while the femoral cutting instrument is located in a femur of the patient.

The femoral neck may be a prosthetic femoral neck of a prosthetic femoral stem and the angle may be visually inspected after the prosthetic femoral stem has been implanted in a femur of the patient.

The orientation guide may be attached to the femoral neck by clipping the orientation guide to the femoral neck.

The orientation guide may be attached to the femoral neck using a tool.

The method may further comprise detaching the tool from the orientation guide after the orientation guide has been attached to the femoral neck.

An embodiment of the invention will now be described in detail, by way of example only, and with reference to the accompanying drawings, in which:

FIG. 1 shows a coronal view of a femur;

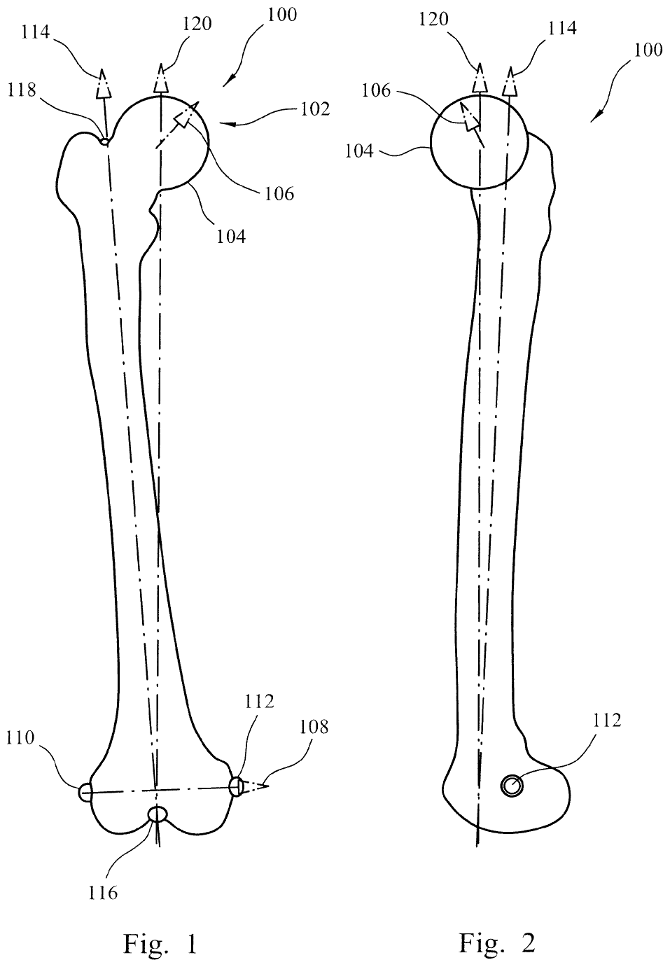

FIG. 2 shows a sagittal view of the femur of FIG. 1;

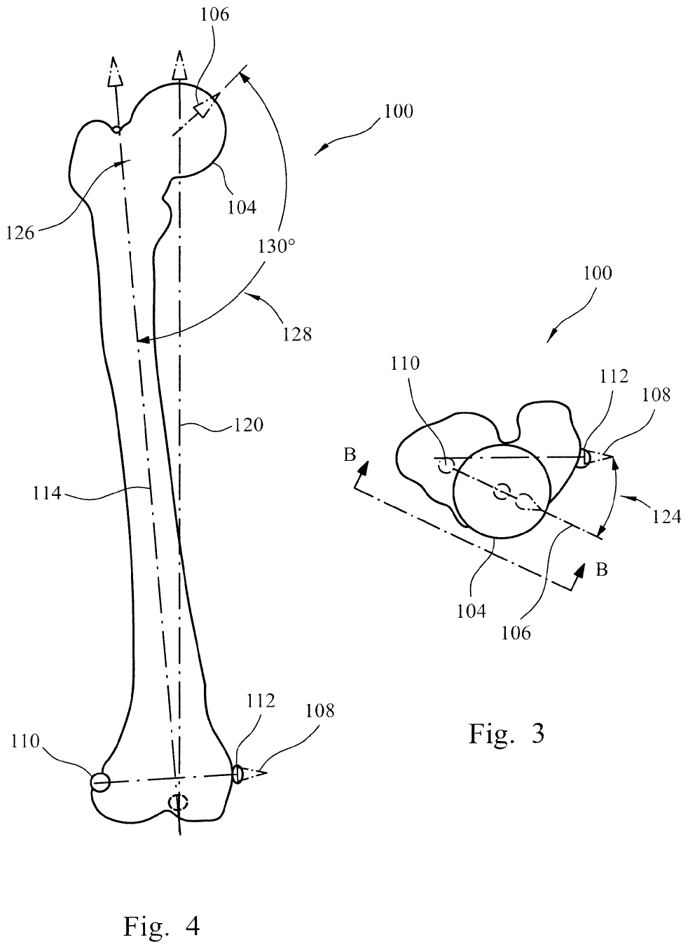

FIG. 3 shows a transverse view of the femur of FIGS. 1 and 2;

FIG. 4 shows a view of the femur in a plane parallel to the anatomic axis and the neck of the femur;

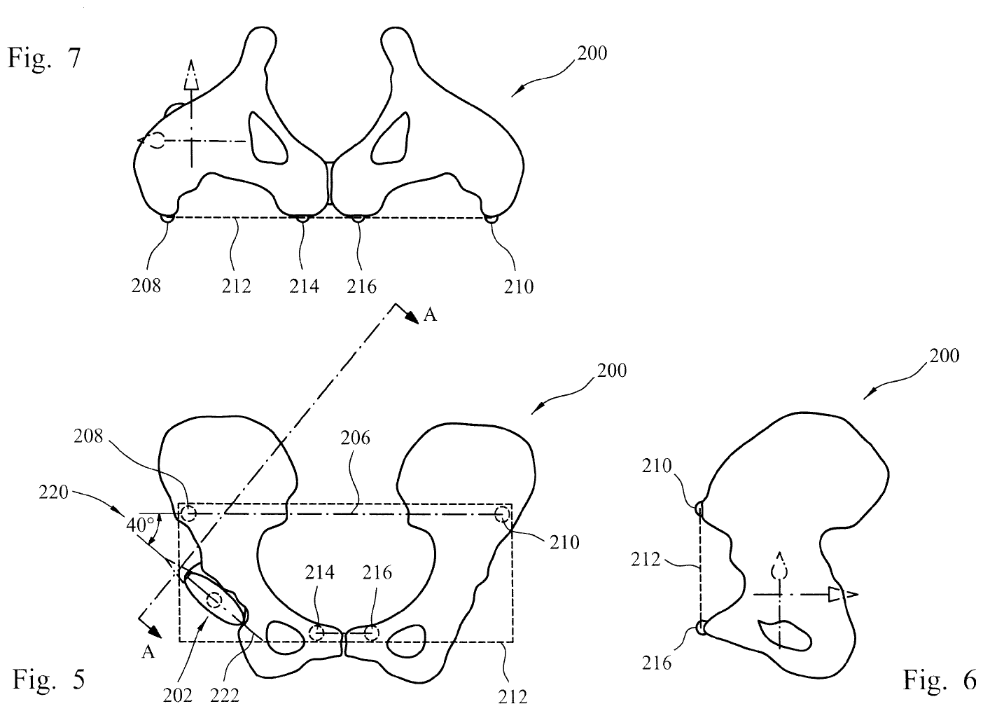

FIG. 5 shows a coronal view of a pelvis;

FIG. 6 shows a sagittal view of the pelvis of FIG. 5;

FIG. 7 shows a transverse view of the pelvis of FIGS. 5 and 6;

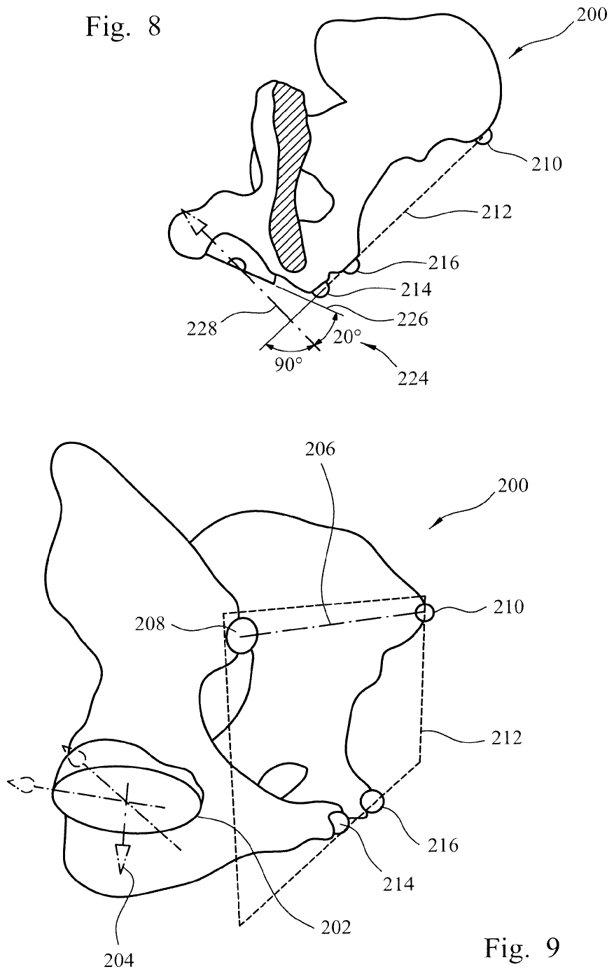

FIG. 8 shows a partial cross sectional perspective view of the pelvis along line A-A of FIG. 5;

FIG. 9 shows a perspective view of the pelvis;

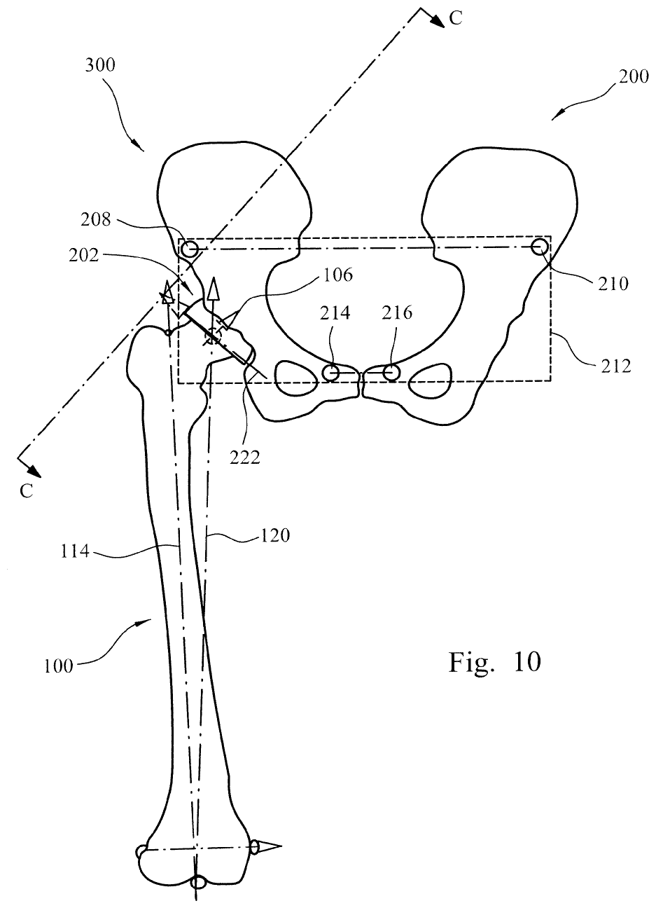

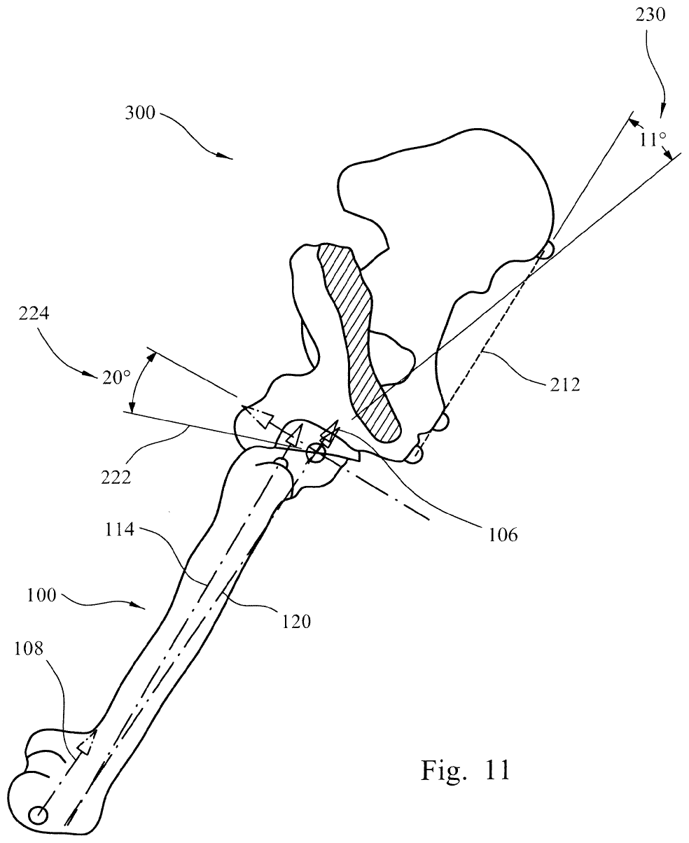

FIG. 10 shows a coronal view of a hip comprising the femur of FIGS. 1 to 4 and the pelvis of FIGS. 5 to 9;

FIG. 11 shows a partial cross sectional perspective view of the pelvis along line C-C of FIG. 10;

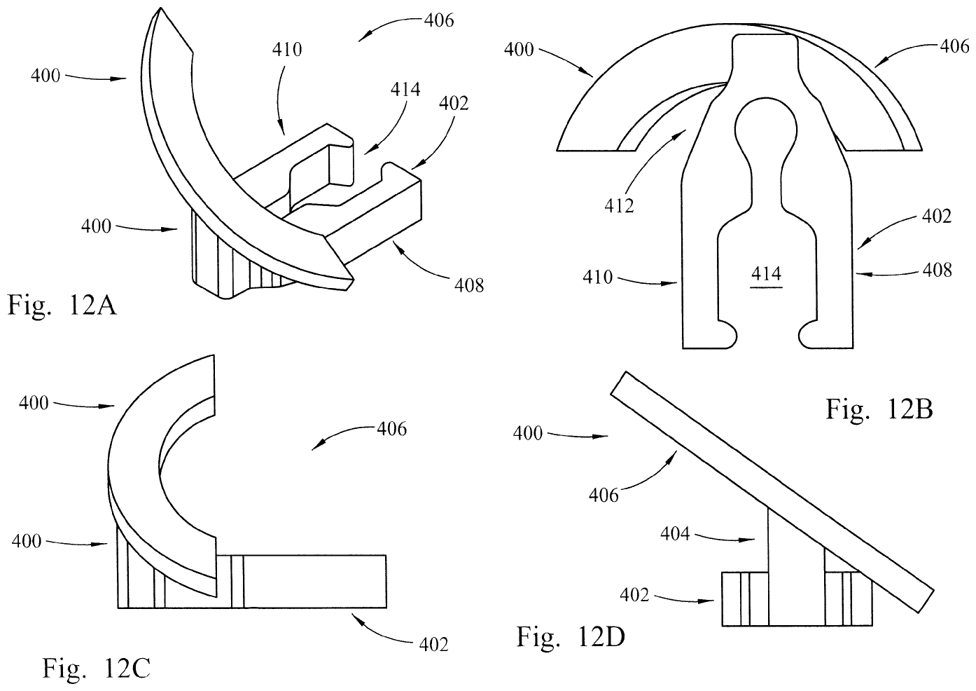

FIGS. 12A to 12D show various views of a first embodiment of an orientation guide according to the invention;

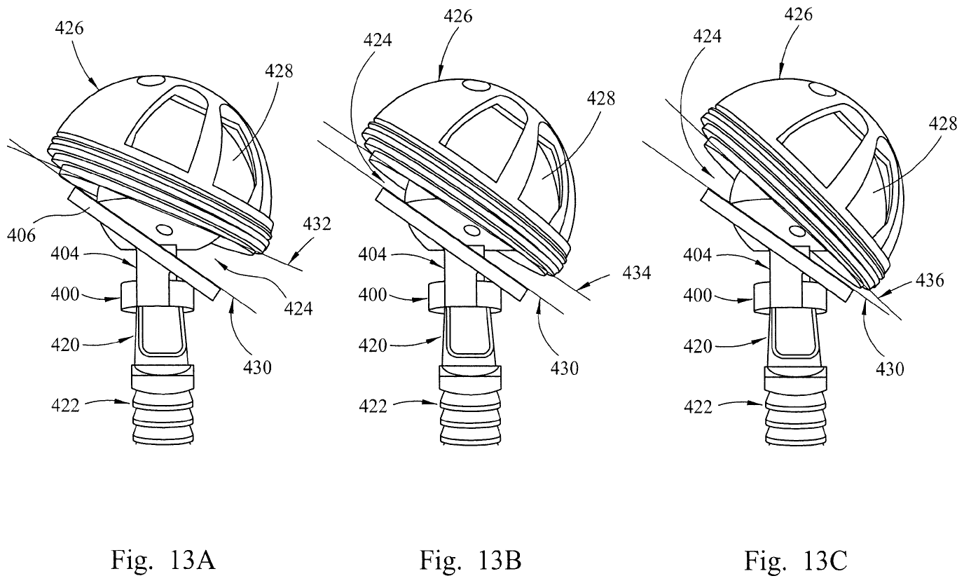

FIGS. 13A to 13C shows views of a hip joint assembly also according to the invention in various configurations and including the orientation guide shown in FIGS. 12A to 12D;

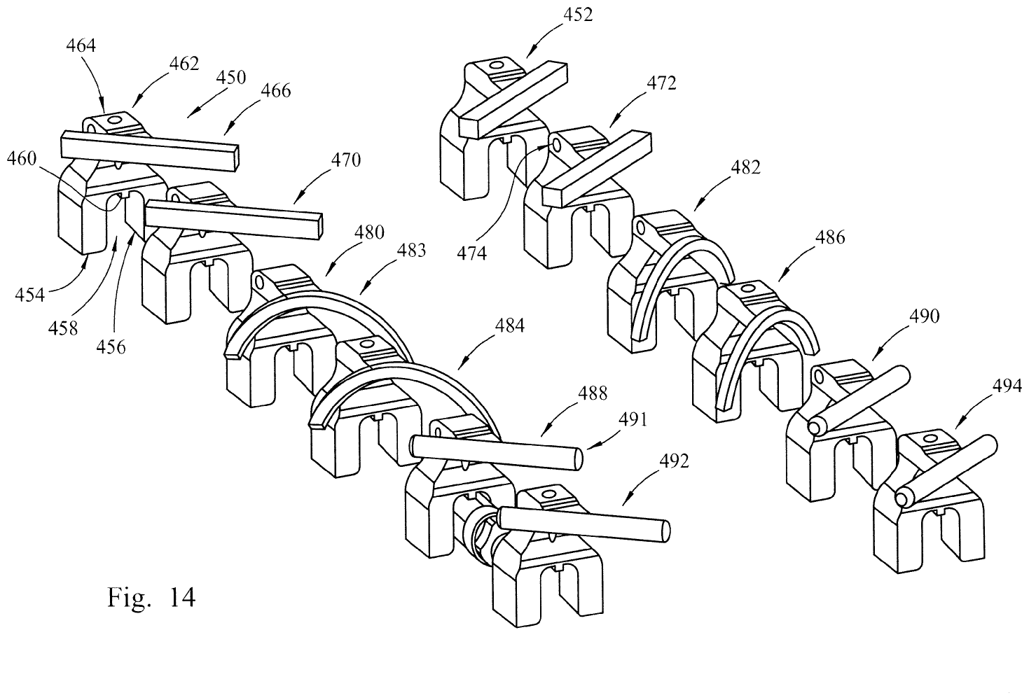

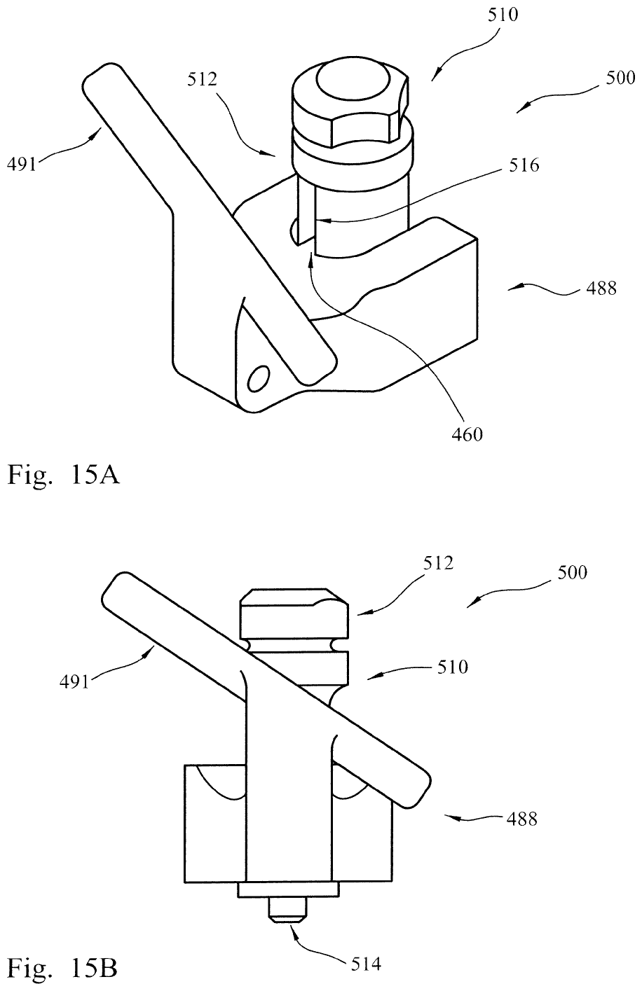

FIG. 14 shows perspective views of pairs of second to seventh embodiments of orientation guides according to the invention;

FIGS. 15A and 15B respectively show perspective and side views of the sixth embodiment of the orientation guide of a pair illustrated in FIG. 14;

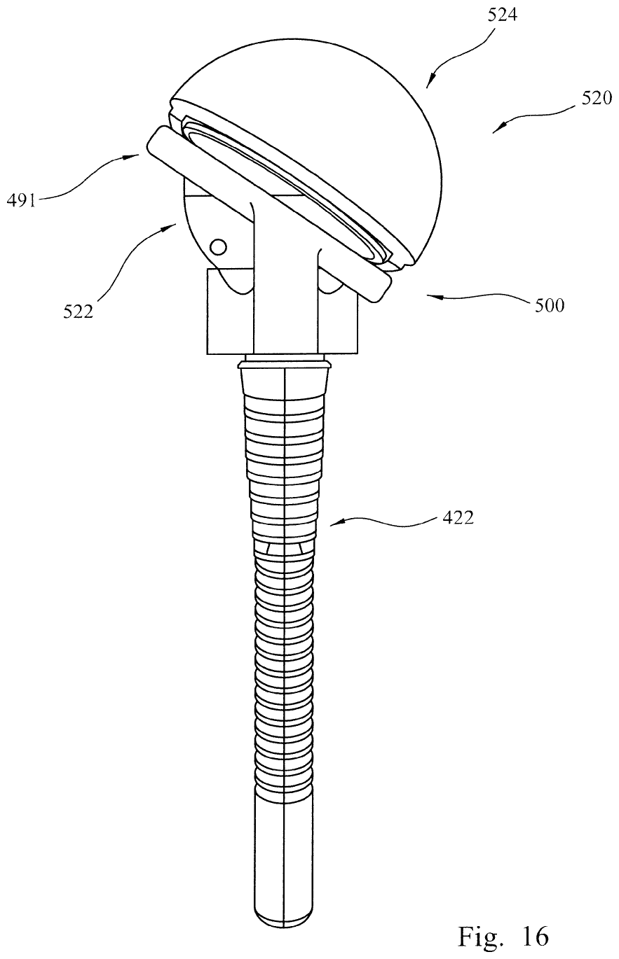

FIG. 16 shows a view of a hip joint assembly also according to the invention and including the sixth embodiment of the orientation guide shown in FIGS. 15A and 15B;

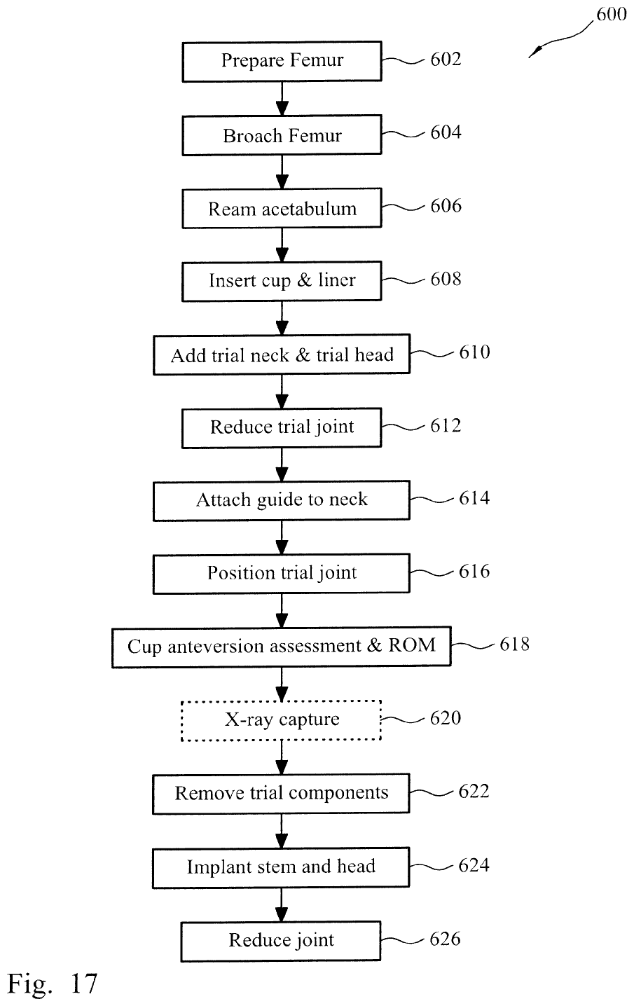

FIG. 17 shows a flow chart illustrating a first embodiment of a method of use of the orientation guide during a hip replacement surgical procedure according to the invention; and

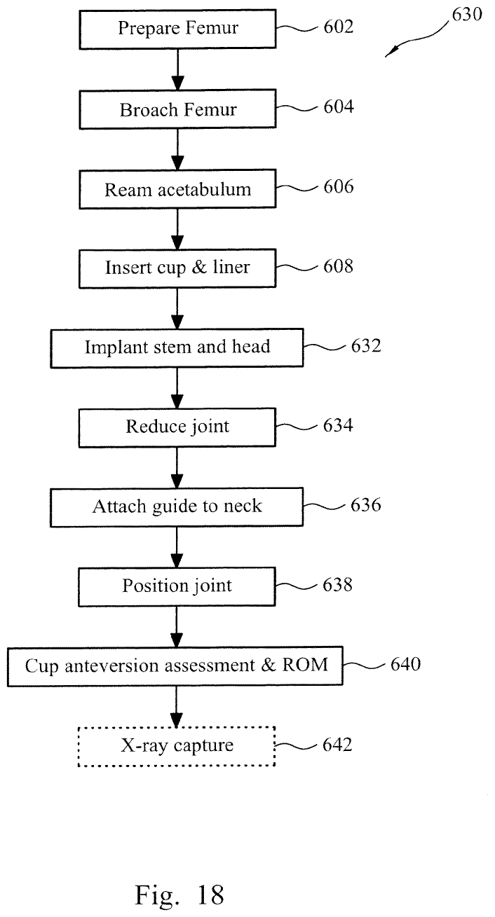

FIG. 18 shows a flow chart illustrating a second embodiment of a method of use of the orientation guide during a hip replacement surgical procedure according to the invention.

Similar items in different Figure shared common reference signs unless indicated otherwise.

Before describing the apparatus and/or methods of the invention, the geometry of a hip joint will be discussed generally. In the below, a right hip joint is described, but it will be appreciated that a similar discussion applies to a left hip joint. Also, the following discussion is intended to relate to both the pre-operative natural, or native, hip joint, as well as to the artificial, or prosthetic, hip joint. Hence, although the magnitude of the various angles may vary between the native hip joint and the prosthetic hip joint, the definitions of those angles may be generally the same for the native and prosthetic hip and may be determined by the positions and/or orientations of the various parts making up the native hip joint and prosthetic hip joint respectively.

With reference to FIGS. 1 to 4, there are shown various different views of a right femur 100. In particular FIG. 1 shows a coronal view in the anterior to the posterior direction (generally herein the AP direction), FIG. 2 shows a sagittal view in the medial to the lateral direction (generally herein the ML direction), FIG. 3 shows a transverse view in the superior to the inferior direction, and FIG. 4 shows a view of the femur in a plane parallel to the anatomic axis of the femur and the neck of the femur as explained in greater detail below. Pre-operatively, the proximal part of the femur 100 includes the native femoral neck and native femoral head. Intra-operatively and post-operatively the proximal part of the femur may include various trial or prosthetic parts or components providing trial or prosthetic femoral necks and femoral heads. The following will refer generally to femoral necks and femoral heads and is intended to include native, trial or prosthetic ones.

With particular reference to FIGS. 1 and 2, the proximal part 102 of femur 100 includes a femoral head 104 (represented by a sphere) attached to a femoral neck 126, best illustrated in FIG. 4, having a neck axis extending generally in the direction of arrow 106. The femur 100 has an epicondylar axis 108 extending between the lateral femoral epicondyle 110 and the medial femoral epicondyle 112. The femur 100 also has an anatomic axis 114 extending between, for example, the distal femur intercondylar notch 116 and the piriformis fossa, close to the medial face of the greater trochanter. The femur 100 also has a mechanical axis 120 extending between, for example, close to the distal femur intercondylar notch 116 and the centre of the femoral head 104. The anatomical axis 114 and mechanical axis 120 of the femur 100 may be defined by other anatomical points in other embodiments.

With reference to FIG. 3, a femoral neck anteversion angle 124 can be defined as the angle in the transverse plane subtended by the femoral neck axis 106 and the epicondylar axis 108. In practice, the femoral neck anteversion angle for the native neck is typically in the range of about 12° to 15°, but may have other values. Neck anteversion angle 124 is a measure of the anteversion of the femoral neck relative to the local anatomy of the femur 100.

FIG. 4 shows a view of the femur 100 in a plane parallel to line BB of FIG. 3, which is parallel to the femoral neck axis 106, and the anatomical axis 114, and which more clearly shows the femoral neck 126. FIG. 4 also illustrates the neck angle 128 subtended between the femoral neck axis 106 and the anatomical axis 114 of the femur. The native neck angle 128 varies from patient to patient, but is typically about 130°. The neck angle 128 for a trial or prosthetic implant is usually fixed by the implant design, unless the implant is adjustable, and is often intended to approximately reproduce the native geometry and so may also be about 130°. In the following a neck angle 128 of 130° may be used as an example, but it will be appreciated that in other embodiments, other neck angle values may also be used.

Hence during hip surgery in which a prosthetic femoral component is used, one of the variables is the femoral neck anteversion angle 124, which generally measures how far forward the femoral neck 126 is directed compared to the medial-lateral axis of the femur.

With reference to FIGS. 5 to 9, there are shown various different views of a pelvis 200 and right acetabulum. In particular FIG. 5 shows a coronal view in the anterior to the posterior direction (generally herein the AP direction), FIG. 6 shows a sagittal view in the medial to the lateral direction (generally herein the ML direction), FIG. 7 shows a transverse view in the superior to the inferior direction, FIG. 8 shows a partial sectional view along line A-A of FIG. 5, and FIG. 9 shows a perspective view of the pelvis 200 with the anterior pelvic plane (APP) vertical and the transverse axis generally horizontal. Pre-operatively, the pelvis 200 includes an acetabulum which provides a native socket in which the native femoral head is received and articulates. Intra-operatively and/or post-operatively the pelvis may include various trial or prosthetic implants, such as trial or prosthetic acetabular cups (with or without liners depending on the specific implant system being used). The following will refer generally to the acetabulum or acetabular cup and is intended to include the native acetabulum as well as trial or prosthetic components.

As illustrated in FIG. 5, the acetabulum 202 may be represented by a hemisphere or hemispherical cup which generally has a position and an orientation. The orientation or direction of the acetabulum may generally be defined by two angles. A first angle indicates how much the acetabulum is directed forward or backward (generally referred to as anteversion when pointing anteriorly and retroversion when pointed posteriorly) relative to the pelvis. A second angle indicates how much the acetabulum is pointing downward or in an inferior direction (generally referred to as inclination or abduction) relative to the pelvis. The direction of the acetabulum may be defined by an acetabular axis 204, best illustrated in FIG. 9, which generally passes through the centre of the mouth of the acetabulum and perpendicular to the plane of the mouth of the acetabulum.

The pelvis 200 includes a transverse axis 206 passing between the right ASIS 208 and the left ASIS 210. An anterior pelvic plane 212 (generally referred to as APP in the following) is defined by the transverse axis 206 and first and second points on the symphysis pubis 214, 216.

As best illustrated in FIG. 5, an inclination angle for the acetabulum or acetabular cup 202 may be defined by the angle 220 subtended by the transverse axis 206 and a long axis, or inclination axis, 222 of the acetabular cup 202 within, or parallel to, the anterior pelvic plane 212. In FIG. 5, the illustrated inclination angle 220 is approximately 40°.

FIG. 8 shows a view of a cross section of the pelvis 200 along line A-A in FIG. 5 and in a direction along the long axis 222 of the acetabular cup 202. Hence, FIG. 8 shows the plane generally perpendicular to the long axis 222 of the pelvic cup 202. From FIG. 8, an anteversion angle 224 may be defined as the angle subtended between the plane 226 of the mouth of the acetabular cup and a plane 228 perpendicular to the anterior acetabular plane 212. Hence, as illustrated in FIG. 8, the acetabulum or acetabular cup 202 has an anteversion angle 224 of approximately 20°.

Hence, as illustrated in the perspective view of the pelvis 200 in FIG. 9, the acetabulum or acetabular cup 202 has an orientation corresponding to an inclination of 40° and an anteversion of 20°. These angles may be referred to as radiographic angles as they are based on the APP view of the pelvis illustrated in FIG. 5 and which is the view of the pelvis typically radiographically imaged or X-rayed and which images are often used by surgeons pre-, intra- and/or post operatively to assess acetabular orientation.

With reference to FIGS. 10 to 11, there are shown various different views of a right hip joint 300 formed by femur 100 and pelvis 200. In particular, FIG. 10 shows a coronal view in the anterior to the posterior direction (generally herein the AP direction), similar to FIGS. 1 and 5 combined. In FIG. 10, the femur has been placed in an anatomical position as described in greater detail below. FIG. 11 shows a partial section along line C-C of FIG. 10 and viewed in a direction along the inclination axis 222 of the acetabular cup 202. Hence, the plane of FIG. 11 is generally perpendicular to the direction of the inclination axis 222 of the acetabular cup.

As illustrated in FIG. 11, the acetabular cup 202 has an anteversion angle 224 of 20° in the plane perpendicular to the inclination axis 222 of the acetabulum. FIG. 11 also illustrates the femoral anteversion angle 230 in the plane perpendicular to the inclination axis of the acetabulum and being defined by the angle 230 subtended in that plane by the femoral neck axis 106 and the anterior pelvic plane 212. As illustrated in FIG. 11, the apparent femoral anteversion angle 230 is approximately 11°.

Hence, as can be seen the overall geometry of the hip joint arises from the orientation of the acetabulum relative to the pelvis and also the orientation of the femoral neck relative to the femur. In the illustrated example, the overall or combined anteversion of the hip joint 200 shown in FIGS. 10 and 11 is the combination of the acetabular anteversion, about 20°, and the amount of femoral anteversion projected into the same plane, which in this example is approximately 11°. Hence, the combined anteversion of the hip joint 300 in this plane is about 31°.

However, in practice, when surgeons talk about a combined anteversion of about 35°, this may be arrived at by adding absolute values of angles in different planes, 15° anteversion of the neck relative to the femur in a first plane and 20° anteversion of the acetabulum relative to the pelvis in a second, different plane, to give approximately 35°. In practice, the combined anteversion is assumed to be in the range of approximately 30° to 40°, as being typically greater than 30° and less than 40°, and that any measurement or assessment is likely to be accurate to plus or minus a few degrees anyway and so measurement of the angles in different, non-parallel planes is not crucial.

Herein, combined anteversion may refer, depending on the context, to the general idea that the anteversion of a hip joint is the combined effect of the degree of anteversion of the femoral neck relative to the femur and also the degree of anteversion of the acetabulum relative to the pelvis. More specifically, for non-extreme cases, combined anteversion may also refer to a general rule of thumb that the sum of the acetabular anteversion and the femoral anteversion, measured in the same plane, should have a certain value, for example approximately 35°. Hence, if a low value of one occurs, then the other can be increased (or vice versa) in order to bring the combined anteversion closer to this target value.

With reference to FIGS. 12A to 12D there are shown various views of a first embodiment of an acetabular cup orientation guide 400 according to the invention. FIG. 12A shows a perspective view of the orientation guide 400, FIG. 12B shows a view of the guide 400 form an under side, corresponding to a generally inferior-superior direction when in use, FIG. 12C shows a view of the guide 400 from a front side, corresponding to a generally anterior-posterior direction when in use and FIG. 12D shows a view of the guide 400 from an end side, corresponding to a generally lateral-medial direction when in use.

The orientation guide 400 comprises a main body 402 and a support 404 extending away from an upper side of the main body and an alignment member 406 mounted on the support 404. The main body 402 includes a first 408 arm and second arm 410 which are joined by a spring portion 412 meet toward the support 404. The first 408 and second 410 arms define an aperture 414 therebetween configured and arranged to receive a femoral neck. The free ends of the first and second arms each include a respective protrusion 416 and 418 to enhance the fastening of the guide to a femoral neck in use. Hence, the sprung arms result in a clip by which the guide 400 can be releasably attached to a part of a femoral neck in use.

The support 404 is in the form of a limb extending away from the body 402 in a direction generally perpendicular to the plane of the main body 402. The alignment member 406 is generally tilted or inclined relative to the plane of the main body and subtends acute an angle of approximately 35° with the plane of the main body 402 (or alternatively subtends an acute angle of approximately 55° with the longitudinal axis of the support 404). The alignment member 406 has a generally curved form and is in the form of an arc of an annulus extending over approximately 120°. The alignment member 406 provides a visual index which can be used to assess the orientation of an acetabular cup. In particular, the alignment member 406 can be used primarily to assess the anteversion of an acetabular cup as described in greater detail below. As the alignment member is aligned to the neck, it can effectively be used to measure anteversion and inclination relative to the broach or stem.

The orientation guide 400 may be made from any suitable biocompatible material such as a metal, alloy or plastic. In particular, the orientation guide may be made from polyphenylsulphone, polyacetal, polyamide, polypropylene, polyarylamide, polyetherimide, acrylonitrile-butadiene-styrene, polymethylmethacrylate, polycarbonate, and the polymers may be unfilled or filled with glass or carbon fibres or beads.

Use of the orientation guide 400 will be briefly described with reference to FIGS. 13A to 13C which show a left hip joint viewed from above. Each of FIGS. 13A to 13C show a view generally in the lateral-medial direction along the neck axis 106 of a trial neck 420, mounted on the superior part of a rasp or broach 422 located within the superior part of a resected femur (not shown), with the guide 400 attached to the trial neck and with a trial femoral head 424 mounted on a taper of the trial neck 420. Also shown is a trial acetabular cup 426 including, optionally, an acetabular liner 428. A prosthetic cup, optionally with a liner, may also be used. The visual alignment member 406 defines a first plane indicated by line 430 in FIGS. 13A to 13C and which is tilted or inclined at an angle of approximately 35° relative to the support 404 and direction of the neck axis 106 in FIGS. 13A to 13C. The mouth of the acetabular cup 426, or liner 428, defines a second plane indicated by line 432 in FIG. 13A, 434 in FIG. 13B and 436 in FIG. 13C.

FIG. 13A shows a reduced trial hip joint generally along the direction of the neck axis of the femoral neck and with the broach 422 is positioned in the femur with a neck anteversion of approximately 15° relative to the femur. The hip joint has been placed in an anatomical configuration or position with respect to the pelvis. Specifically, the femur is placed in 0° of flexion/extension, 0° of adduction/abduction and 0° of internal/external rotation. This is described in further detail below.

In FIG. 13A the acetabular cup 426 has been placed in the pelvis with an orientation of approximately 40° inclination and approximately 10° anteversion relative to the pelvis. The inclination of the alignment member 406 has been configured to correspond to a cup anteversion of approximately 20° relative to the pelvis, and hence a combined anteversion of approximately 35°. Hence, as illustrated in FIG. 13A the plane of the mouth of the acetabular cup 432 is not parallel to the plane of the alignment member 430 which provides a visual indication of the anteversion of the cup. In particular, in the described embodiment, the orientation guide 400 provides a visual indication of how closes the anteversion of the acetabular cup is to a target cup anteversion angle of 20° relative to the pelvis, or combined anteversion angle of the hip joint of approximately 35°. In FIG. 13A, the combined anteversion angle is approximately 25° and the extent to which the mouth of the acetabular cup and alignment member are parallel indicates how far from the target anteversion angle the cup is.

The partially-annular component 406 defines a plane and hence allows both inclination and anteversion of the cup to be assessed. However in other embodiments, as describe below, the alignment member may be used to define an axis or line only and which are suitable for assessing anteversion only. For example FIG. 14 shows various embodiments with alignment members defining a plane or an axis. As described below, an alignment member in the form of a round bar may be used to assess anteversion only. An alignment member in the form of a square bar may be used mainly to assess anteversion but may also help to assess inclination. Whereas the alignment member in the form of a semi-annulus indicates a plane which may be used to asses anteversion or inclination or a combination thereof.

Returning to FIGS. 1A to 13C, in FIG. 13B the acetabular cup 426 has been placed in the pelvis with an orientation of approximately 40° inclination and approximately 20° anteversion relative to the pelvis. Hence, for the cup orientation illustrated in FIG. 13B the plane of the mouth of the acetabular cup 434 is generally parallel to the plane of the alignment member 430 and which provides a visual indication of the anteversion of the cup. In particular, the orientation guide 400 provides a visual indication that the anteversion of the acetabular cup is close to the target cup anteversion angle of 20° relative to the pelvis, or a combined anteversion angle of the hip joint of approximately 35°.

In FIG. 13C the acetabular cup 426 has been placed in the pelvis with an orientation of Hence, as illustrated in FIG. 13C the plane of the mouth of the acetabular cup 436 is not parallel to the plane of the alignment member 430 which provides a visual indication of the anteversion of the cup. In FIG. 13C, the combined anteversion angle is approximately 45° and the extent to which the mouth of the acetabular cup and alignment member are parallel indicates how far from the target anteversion angle the cup is.

The first embodiment of the orientation guide 400 shown in FIGS. 12A to 12D is particularly suitable for sue with a trial neck and can simply be clipped in place on a trial neck during a trialling stage of hip surgery, as described in greater detail below. Before doing so, further embodiments of the orientation guide of the invention will be described with reference to FIG. 14.

FIG. 14 shows perspective views of six pairs of orientation guides, each pair including an orientation guide for a left hip and a right hip.

A second embodiment of the orientation guide for a left hip 450 and a right hip 452 is shown in FIG. 14 and is generally similar to the first embodiment 400. However, in the second embodiment, the pair of arms 454, 456 defining the aperture 458 for receiving the femoral neck are not sprung, but are rigid. A rib 460 is provided by the body and extending into the aperture 458 and is generally aligned with the support 462. Support 462 also includes a female formation 464 for receiving a male formation for a tool or instrument which can be used to position the orientation guide on a femoral neck. For example, the female formation may be a threaded bore which can receive a threaded free end of a placement tool or instrument having a handle (not shown). In the second embodiment, the tool attachment feature 464 is directed generally in the lateral-medial direction of the orientation guide in use. Hence, the second embodiment is particularly suitable for use in a posterior-lateral approach in which the patient is in the lateral decubitus position.

Also, in the second embodiment, the alignment member 466 is generally in the form of a rectilinear or straight bar with a generally square cross sectional shape which may be used primarily to assess anteversion and secondly to assess inclination.

The right hip orientation guide 452 is generally similar to the left hip orientation guide 450, but is generally a mirror image thereof.

A third embodiment of the orientation guide for a left hip 470 and a right hip 472 is shown in FIG. 14 and is generally similar to the second embodiment 450, 452. In the third embodiment, the tool attachment feature 474 is directed generally in the anterior-posterior direction of the orientation guide in use. Hence, the third embodiment is particularly suitable for use in an anterior approach when a patient is in the supine position.

A fourth embodiment of the orientation guide for a left hip 480 and a right hip 482 and a fifth embodiment of the orientation guide for a left hip 484 and a right hip 486 are shown in FIG. 14 and are respectively generally similar to the second and third embodiments. However, in the fourth and fifth embodiments, the alignment member 483 is in the form of an annular arc similar to the alignment member 406 of the first embodiment.

A sixth embodiment of the orientation guide for a left hip 488 and a right hip 490 and a seventh embodiment of the orientation guide for a left hip 492 and a right hip 494 are shown in FIG. 14 and are respectively generally similar to the second and third embodiments. However, in the sixth and seventh embodiments, the alignment member 491 is in the form of a rectilinear or straight bar with a generally circular cross sectional shape and which may be used to assess anteversion only.

The pairs of orientation guides of the second, 450, 452, fifth 484, 486 and seventh 492, 494 embodiments, having the tool attachment formation 464 extending generally in the lateral-medial direction in use and particularly suitable for use in a posterior approach when the patient is in a lateral decubitus position.

The pairs of orientation guides of the third 470, 472, fourth 480, 482, and sixth 488, 490 embodiments, having the tool attachment formation extending generally in the anterior-posterior direction in use are particularly suitable for use in an anterior approach when the patient is supine on the operating table.

The orientation guides shown in FIG. 14 may be made from any suitable biocompatible material such as a metal, alloy or plastic. In particular, the alignment guides may be made from polyphenylsulphone, polyacetal, polyamide, polypropylene, polyarylamide, polyetherimide, acrylonitrile-butadiene-styrene, polymethylmethacrylate, polycarbonate, and the polymers may be unfilled or filled with glass or carbon fibres or beads.

FIG. 15A shows a perspective view of an assembly 500 of the sixth embodiment of the orientation guide 488 and a trial femoral neck 510 and FIG. 15B shows a view in a generally lateral-medial direction of the assembly 500. As illustrated in FIG. 15A, the trial neck 510 includes a taper 512 at a free end for releasably attaching a trial femoral head. The trial neck 510 also includes at least one formation 514 for releasably attaching the trial neck 510 to a femoral part 422, such as a broach or a rasp. In particular, the trial neck 510 includes a male attachment feature 514 in the form of a circular peg and an aperture (not visible in FIG. 15B) for receiving a corresponding male feature in a superior part of the broach or rasp. As best illustrated in FIG. 15A, the trial neck 510 includes a groove 516 extending generally along the trial neck axis on a superior side of the trial neck and into which the rib 460 of the orientation guide 488 can be received. The rib 460 and groove 516 co-operate to prevent rotation of the orientation guide about the trial neck and also help to avoid accidental attachment of the orientation guide to the underside of the trial neck.

FIG. 16 shows a view of a trail hip joint 520 for a left hip including the assembly 500 and generally in the same direction as FIG. 15B. The trial hip joint 520 includes the orientation guide 488 mounted on the trial neck which is mounted on the femoral rasp or broach 422. A trial femoral head 522 is mounted on the trial neck and the trial femoral head 522 is received within the cavity of an acetabular cup 524. FIG. 16 shows the trial hip joint 520 generally in the lateral-medial direction along the femoral neck axis which in practice may be anteverted relative to the femur. The alignment member 491 is configured to correspond to a cup anteversion angle of approximately 20° relative to the pelvis, and hence to a combined anteversion of approximately 35°, when the femoral part 422 is anteverted by approximately 15° relative to the femur and when the femur is placed to put the trial hip joint into the anatomical position discussed above. Hence, in FIG. 16, the orientation guide provides a visual indication that the acetabular cup has been placed with a target anteversion of approximately 20° relative to the pelvis as the plane of the mouth of the acetabular cup 524 is generally parallel to the alignment member 491.

Although FIGS. 15A, 15B and 16 show the orientation guide in use with a trial neck, the orientation guide of the invention can also be used with the neck of a prosthetic femoral stem.

With reference to FIG. 17, there is shown a flow chart illustrating a hip replacement surgical procedure 600 in which the orientation guide and assembly 500, may be used. Many of the steps are similar when the orientation guide is used with a trial neck or a prosthetic neck. A trial neck method will be described first. The order of some of the steps is not relevant but the order of some of the steps may be relevant as will become apparent from the following description. Also, some of the described steps may be optional, and may be omitted, depending on the workflow that a surgeon may prefer.

At 602, the femur is prepared in a generally conventional manner which may include resecting the native femoral neck and head and then at 604 a cavity is formed along the intramedullary canal to accept a femoral stem component and which typically includes using one or more broaches and/or rasps. When a final sized broach has been used, then a broach handle is removed and the broach is left in the femur. At 606, the acetabulum is prepared in a generally conventional manner which may include removing soft tissue and forming a hemispherical cavity within the native acetabulum using an acetabular reamer. At 608 a prosthetic acetabular cup is inserted in the acetabular cavity by the surgeon using a cup inserter and with a certain orientation (version and abduction). Any cup liner may also be inserted in the implanted acetabular cup at 608.

At 610, the trial neck 510 is attached to the broach 422 and a trail femoral head 522 is attached to the trial neck 510. At 612, the trial joint is reduced by introducing the trial femoral head 522 into the implanted acetabular cup 524, or liner. The orientation guide is then attached to the trial neck at 614. Depending on the embodiment being used, the orientation guide may simply be clipped in place on the trial neck by hand or a tool or instrument may be attached to the orientation guide and then used by the surgeon to slide the orientation guide onto the trial neck. Then at 616, the patient's pelvis and/or leg are manipulated to place the trial joint in the preselected position or configuration.

In particular, the patient's leg is placed with the femur in an anatomical position with respect to the pelvis. Specifically, the femur is placed in 0° of flexion/extension, 0° of adduction/abduction and 0° of internal/external rotation.

Assuming that the pelvis on the operating table has taken up an approximately neutral amount of pelvic tilt then this can be achieved by comparing the following three factors. The long axis of the leg relative to the longitudinal axis of the patient can be adjusted by pulling lightly on the lower limb to pull the leg into full extension. If an anterior approach is being used, with the patient supine, then the leg will naturally rest in full extension. Zero abduction/adduction of the legs can be achieved by placing both lower legs (tibia from knee to ankle) parallel and almost touching. An internal/external rotation angle of 0° can be checked using the epicondyle axis of the knee relative to the transverse axis of the pelvis. If the pelvis is level (supine) or vertical (in a lateral decubitus approach) on the table, then the epicondyle axis can be compared to the table. If a posterior approach is being used, with the patient lateral, then the tibia can be flexed 90° to provide a more discernible indication on the femoral articular axis which will indicate any internal/external rotation of the femur. Hence, depending on the orientation of the patient on the table, the patient's legs are placed in appropriate positions to provide 0° of flexion/extension, 0° of abduction/adduction and 0° of internal/external rotation of the femur with respect to the pelvis. With the patient's legs in this anatomical position, an assessment of the trial cup position can be carried out at step 618.

If the cup has been placed with an orientation having an anteversion relative to the pelvis corresponding to the value to which the alignment member has been configured, e.g. 20°, then as illustrated in FIG. 13B, the alignment member 406 will be generally parallel to the rim of the cup, or liner. Alternatively, if the cup has been placed with an orientation having an anteversion different to the target value, then the rim alignment member 406 will be not be parallel to the rim of the cup, or liner, as illustrated in FIGS. 13A and 13C. Hence, the alignment member 406 provides a visual indication of the anteversion angle of the acetabular cup, as the angle subtended between the alignment member 406 and plane of the rim of the cup, or liner, indicates generally how far away angularly the cup has been placed compared to the target value. Hence, at 618 the surgeon may assess the cup anteversion by visually inspecting the trial joint assembly to see how close the alignment member 406 is to parallel to the plane of the mouth of the cup or liner.

Optionally, at step 618 a range of motion (ROM) assessment can also be carried out. This is generally known in the art and involves articulating the trial hip joint to detect the likely range of articulation possible before impingement of the hip components.

At 620 an intra-operative X-ray image of the trial joint in the anterior-posterior direction (corresponding to FIG. 5) may be captured with the trial joint in the preselected position.

It is not necessary that the surgeon carryout any repositioning of the cup. The trial assessment at 618 may be carried out simply to assess the anteversion angle achieved so as to provide so immediate intra-operative feedback of the cup orientation to the surgeon.

At 622, all the trial femoral components, including the broach, are removed from the femur. Then at 624, the actual prosthetic femoral stem is implanted in the femoral cavity and the prosthetic femoral head is attached. Then at 626, the joint can be reduced and any range of motion trial carried out if desired. The surgical procedure then substantially ends.

The overall method 630 is generally similar when the prosthetic neck is used instead of a trail neck. However, in this case the prosthetic stem and neck are implanted at 632 and then the prosthetic joint is reduced at 634. Then, the orientation guide is attached to the prosthetic stem neck at 636 before the prosthetic hip joint is placed in the anatomical position at step 638. The cup anteversion angle, and combined anteversion of the prosthetic hip joint, can then be assessed at 640 by visually inspecting how parallel the alignment member 406 and the plane of the mouth of the cup, or liner, are and which provides an indication of the anteversion of the cup relative to the pelvis and also how far from the target anteversion the cup has been placed. Optionally, an X-ray may be captured at step 642 before or after the end of the surgical procedure.

Each of methods 600 and 630 may generally be used for the patient in a supine position or a lateral decubitus position.

Indeed, the orientation guide of the invention can generally be used to check the combined anteversion with a patient in the supine position or the lateral decubitus position. Hence, the present invention provides a more widely usable anteversion assessment, compared to previous approaches, such as the Ranawat sign approach which can only be used with the patient in a supine position.

In this specification, example embodiments have been presented as particular combinations of features. However, a person of ordinary skill in the art would understand that many other embodiments may be practiced which include a different combination of features, including fewer features or a greater number of features. It is intended that the following claims cover all possible embodiments.

Any instructions and/or flowchart steps may be carried out in any order, unless a specific order is explicitly stated or would be understood to be required from the context of the description. Also, those skilled in the art will recognize that while example methods have been discussed, a variety of other differing methods are possible based on other combinations and/or orders of method steps, and are to be understood within the context provided by this detailed description.

While the inventions are amenable to various modifications and alternative forms, specific embodiments are shown by way of example in the drawings and described in detail. It should be understood, however, that other embodiments, beyond the specific embodiments described, are possible as well. All modifications, equivalents, and alternative embodiments falling within the scope of the appended claims are covered as well.

1. An orientation guide for visually assessing the orientation of an acetabular cup implanted in a patient, the orientation guide comprising:
a body adapted to be mounted on a femoral neck;
a support extending from the body; and
an alignment member mounted on the support and wherein the alignment member is tilted relative to the body and configured to extend along an alignment axis parallel to a target anteversion angle of the acetabular cup when the orientation guide in use is attached to the femoral neck and the femoral neck includes a femoral head which is received in the acetabular cup to form a hip joint.

2. The orientation guide of claim 1, wherein the alignment member is rectilinear and extends in a straight line along the alignment axis.

3. The orientation guide of claim 1, wherein the alignment member extends over an alignment plane which includes the alignment axis.

4. The orientation guide of claim 3, wherein the alignment member is curved.

5. The orientation guide of claim 4, wherein the alignment member is an arc of an annulus.

6. The orientation guide of claim 1, wherein the body includes a first leg and a second leg and wherein the first leg and second leg define a cavity configured to receive a femoral neck in use.

7. The orientation guide of claim 6, wherein the first leg and/or the second leg are sprung to provide a clip for releasably attaching the orientation guide to the femoral neck in use.

8. The orientation guide of claim 1 and further including a tool attachment feature for releasably attaching a tool for placing the orientation guide on the femoral neck.

9. The orientation guide of claim 8, wherein the tool attachment feature is positioned in a lateral-medial direction or an anterior-posterior direction in use.

10. The orientation guide of claim 1, wherein the target anteversion angle of the acetabular cup is 20° relative to the patient's pelvis.

11. The orientation guide of claim 1, wherein the hip joint is in an anatomical position corresponding to the patient's femur being placed in 0° of flexion/extension, 0° of adduction/abduction and 0° of internal/external rotation.

12. The orientation guide of claim 1, wherein the alignment member is configured for assessing the anteversion of the acetabular cup.

13. The orientation guide of claim 12, wherein the alignment member is configured for assessing the inclination of the acetabular cup.

14-26. (canceled)
